(19) United States
(12) Patent Application Publication
(10) Pub. No.: US 2023/0255746 A1
(43) Pub. Date: Aug. 17, 2023

(54) SYSTEM AND METHOD FOR REPAIRING A TENDON

(71) Applicant: Pedro Juan LOREDO, Southlake, TX (US)

(72) Inventor: Pedro Juan LOREDO, Southlake, TX (US)

(21) Appl. No.: 17/671,676

(22) Filed: Feb. 15, 2022

(51) Int. Cl. A61F 2/08

(52) U.S. Cl. CPC A61F 2/0811; A61F 2002/0835; A61F 2002/0864

(57) ABSTRACT

An anchor for repairing a tendon having a first free end positioned adjacent a second free end to form an adjoined section of tendon is provided. The anchor includes a first anchor section having a fastening mechanism. The fastening mechanism of the first anchor section is configured to secure to a second anchor section to secure the first free end of tendon to the second free end of tendon.

### CROSS-REFERENCE TO RELATED APPLICATIONS

This application claims priority under 35 U.S.C. § 119(e) to U.S. Provisional Application No. 63/286,212, filed Dec. 6, 2021, which is expressly incorporated by reference herein.

### TECHNICAL FIELD

The present invention is directed to a system and method for repairing a tendon and, more particularly, to a system and method of repairing a tendon using anchors.

### BACKGROUND

Tendon injuries are a common occurrence and frequently happen when the tendon is torn. In some cases, the tendon is completely torn so that a first section of the tendon freely extends from a muscle and a second section of tendon freely extends from an adjacent bone. To repair the tendon, the damaged ends of each section are generally sutured together and secured until the tendon is repaired when the sections of the tendon naturally reattach to one another. Unfortunately, suturing the tendon can be difficult and tedious and may result in future tears that reinjure the tendon.

### SUMMARY

The present disclosure includes one or more of the features recited in the appended claims and/or the following features which, alone or in any combination, may comprise patentable subject matter.

According to a first aspect of the disclosed embodiments, a method of repairing a tendon having a first free end and a second free end includes positioning the first free end of tendon adjacent the second free end of tendon to form an adjoined section of tendon. The method also includes positioning a first anchor section against a first side of the adjoined section of tendon. The method also includes positioning a second anchor section against a second side of the adjoined section of tendon so that the second anchor section is aligned with the first anchor section. The method also include securing the first anchor section to the second anchor section to secure the first free end of tendon to the second free end of tendon.

In some embodiments of the first aspect, securing the first anchor section to the second anchor section may include securing a fastening mechanism of the first anchor section to the second anchor section. Securing a fastening mechanism of the first anchor section to the second anchor section may include securing a tine of the first anchor section to the second anchor section. Securing a tine of the first anchor section to the second anchor section may include embedding the tine of the first anchor section into the second anchor section. The second anchor section may be configured to plastically deform when the first anchor section is secured to the second anchor section. Securing the first anchor section to the second anchor section may include extending a portion of the first anchor section through the adjoined section of tendon. Extending a portion of the first anchor section through the adjoined section of tendon may include extending the portion of the first anchor section through the first free end of tendon and the second free end of tendon. The method may also include extending the portion of the first anchor section through the second anchor section. Securing the first anchor section to the second anchor section may include squeezing the first anchor section and the second anchor section together.

According to a second aspect of the disclosed embodiments, an anchor for repairing a tendon having a first free end positioned adjacent a second free end to form an adjoined section of tendon is provided. The anchor includes a first anchor section having a fastening mechanism. The first anchor section is configured to position on a first side of the adjoined section of tendon. A second anchor section is configured to position on a second side of the adjoined section of tendon. The fastening mechanism of the first anchor section is configured to secure to the second anchor section to secure the first free end of tendon to the second free end of tendon.

In some embodiments of the second aspect, the fastening mechanism may be a tine that is configured to secure to the second anchor section. The second anchor section may be formed from a plastic material and the tine may be configured to embed in the plastic material. A plurality of tines may embed in the plastic material. The first anchor section and the second anchor section may be formed as rings. The first anchor section may include a ring shaped body. A plurality of fastening mechanisms may extend from the ring shaped body. The fastening mechanism of the first anchor portion may be configured to extend through the adjoined section of tendon.

According to a third aspect of the disclosed embodiments, an anchor for repairing a tendon having a first free end positioned adjacent a second free end to form an adjoined section of tendon is provided. The anchor includes a first anchor section. The first anchor section is configured to position on a first side of the adjoined section of tendon. A second anchor section is formed from a plastic material and is configured to position on a second side of the adjoined section of tendon. A portion of the first anchor section is configured to embed in the second anchor section to secure the first free end of tendon to the second free end of tendon.

In some embodiments of the third aspect, a plurality of portions of the first anchor section may be configured to embed in the second anchor section. The portion of the first anchor section may be configured to extend through the adjoined section of tendon. The first anchor section and the second anchor section may be formed as rings.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of an anchor for repairing a damaged tendon, wherein the anchor includes a first anchor section and a section anchor section;

FIG. 2 is a perspective view of the anchor inserted in a pair of pliers so that the anchor can be secured to the damaged tendon;

FIG. 3 is a perspective view of the pliers in a closed position securing the anchor to the damaged tendon;

FIG. 4 is a perspective view of the anchor secured to the damaged tendon;

FIG. 5 is a rear view of a leg having a damaged Achilles tendon, wherein the Achilles tendon is separated into a proximal free end and a distal free end;

FIG. 6 is a rear view of the leg, wherein the proximal free end of the damaged tendon is overlapped onto the distal free end of the tendon;

FIG. 7 is a rear view of the leg, wherein the anchor secures the proximal free end of the tendon to the distal free end of the tendon;

FIG. 8 is a top view of another embodiment for repairing a tendon using the anchor shown in FIG. 1; and

FIG. 9 is a top view of yet another embodiment for repairing a tendon using the anchor shown in FIG. 1.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

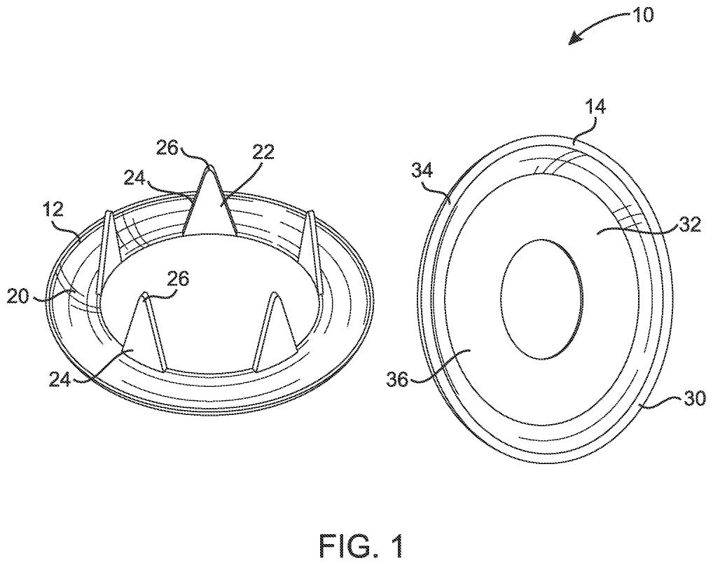

Referring now to FIG. 1, an anchor 10 for repairing a damaged tendon is provided. In an exemplary embodiment, the tendon is torn into a first section having free end extending from a muscle and a second section having a free end extending from an adjacent bone. The free ends are adjoined adjacent one another and secured to one another with the anchor 10. The anchor 10 includes a first anchor section 12 and a section anchor section 14. The first anchor section 12 is configured to secure to the second anchor section 14 to secure the anchor 10 to the tendon. In the illustrated embodiment, the first anchor section 12 and the second anchor section 14 are configured as rings. However, it should be noted that the first anchor section 12 and the second anchor section 14 may take any reasonable shape for repairing the tendon, such as circular, squared, etc.

The first anchor section 12 includes a body 20 illustrated as a ring. At least one fastening mechanism 22 extends outward from the body 20. In the illustrated embodiment, the first anchor section 12 includes a plurality of fastening mechanisms 22. Each of the plurality of fastening mechanisms 22 extends in the same direction from the body 20. The plurality of fastening mechanisms 22 are evenly spaced around the body 20. In an exemplary embodiment, the fastening mechanisms 22 includes tines 24 having pointed ends 26.

The second anchor section 14 includes a body 30 that is illustrated as being ring shaped. At least a portion of the body 30 includes a plastic material 32 that is configured to receive the tines 24 of the first anchor section 12. That is, when the first anchor section 12 is secured to the second anchor section 14, the tines 24 embed within the plastic material 32. In the exemplary embodiment, the entire second anchor section 14 is formed from the plastic material 32. In other embodiments, a portion 34 of the body 30 may be formed from a non-plastic material, for example, metal, and another portion 36 of the body 30 may be formed from the plastic material 32.

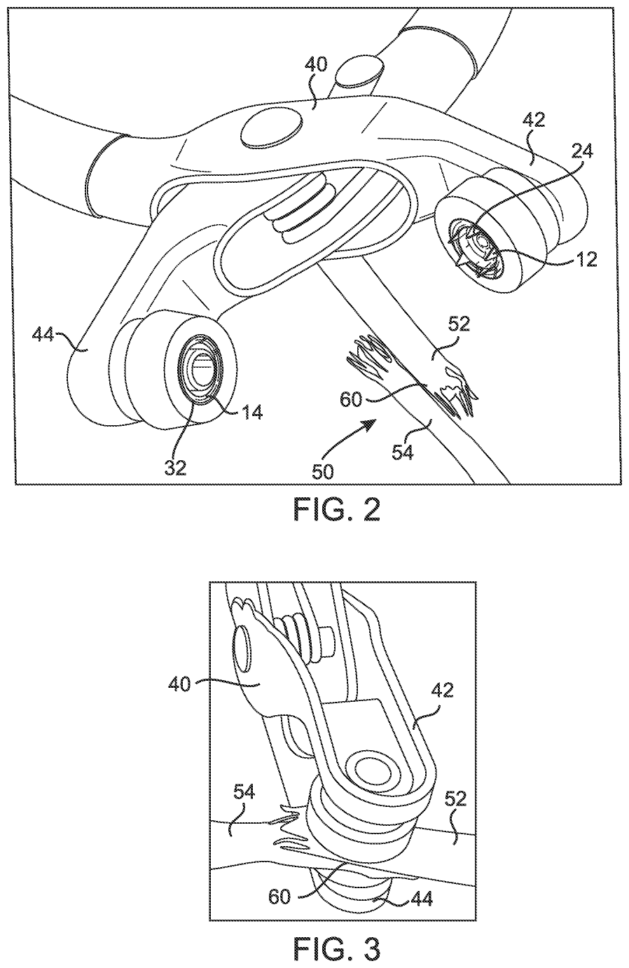

As illustrated in FIG. 2, the first anchor section 12 and the second anchor section 14 are configured to be positioned within a pair of pliers 40. That is, the first anchor section 12 is positioned in a first arm 42 of the pliers 40, and the second anchor section 14 is positioned in a second arm 44 of the pliers 40. During use, the pliers are positioned around the tendon 50 wherein the first section 52 of the tendon 50 is overlapped with the second section 54 of the tendon 50 to form an overlapped tendon 60.

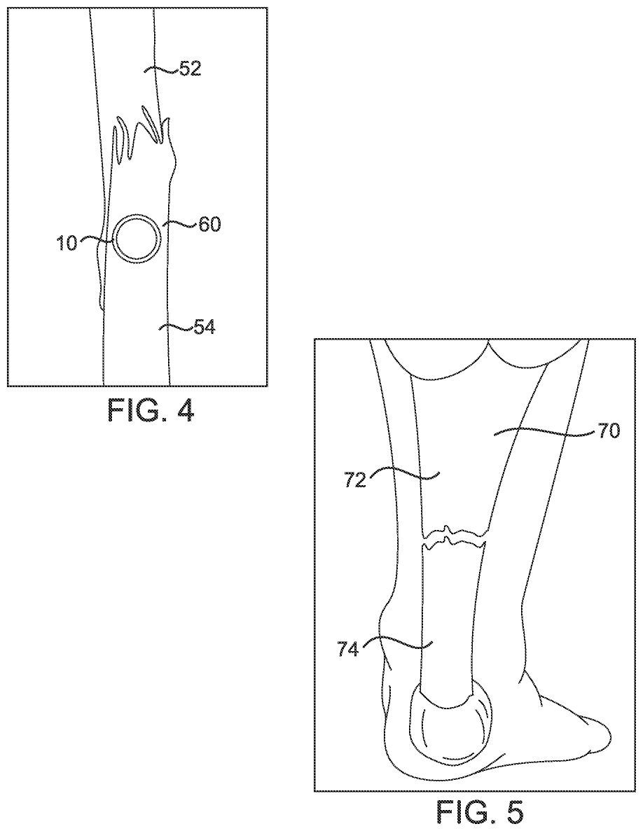

As illustrated in FIG. 3, the first arm 42 of the pliers 40 having the first anchor section 12 therein is positioned on a first side 62 of the overlapped tendon 60. The second arm 44 of the pliers having the second anchor section 14 therein is positioned on a second side 64 of the overlapped tendon 60. By squeezing the first arm 42 and the second arm 44 of the pliers 40 together, the tines 24 are secured through the overlapped tendon 60 and embed in the second anchor section 14, as illustrated in FIG. 4. That is, the tines 24 of the first anchor section 12 extend through the first section 52 and second section 54 of tendon 50 and embed in the plastic material 32 of the second anchor section 14 to secure the first section 52 of tendon 50 to the second section 54 of tendon 50. It will be appreciated that the plastic material 32 plastically deforms to receive the tines 24.

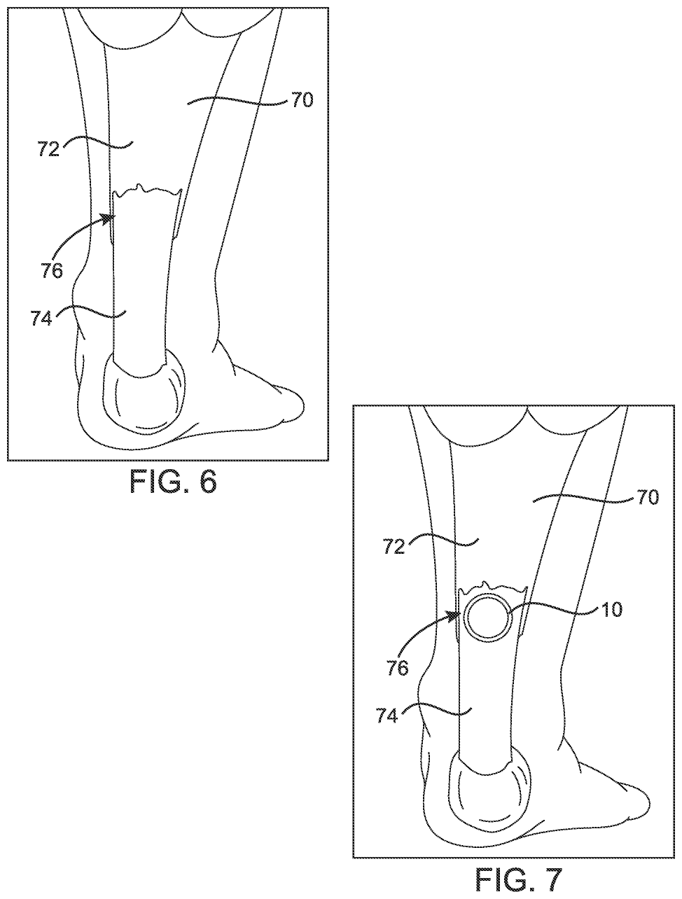

FIGS. 5-7 illustrate a method for repairing an Achilles tendon 70 that has been torn leaving a proximal free end 72 extending from muscle and a distal free end 74 extending from bone, as illustrated in FIG. 5. Although, FIGS. 5-7 are discussed with relation to repairing the Achilles tendon 70, it will be appreciated that the method described herein and the anchor 10 described herein may be used to repair any tendon. As illustrated in FIG. 6, the proximal free end 72 of the Achilles tendon 70 and the distal free end 74 of the Achilles tendon 70 are overlapped to form an overlapped section 76 of tendon 70. The first anchor section 12 is positioned against a first side of the overlapped section 76 of tendon 70, and the second anchor section 14 is positioned against a second side of the overlapped section 76 of tendon 70 so that the second anchor section 12 is aligned with the first anchor section 14.

As illustrated in FIG. 7, the first anchor section 12 is secured to the second anchor section 14 to secure the proximal free end 72 of the Achilles tendon 70 to the distal free end 74 of the Achilles tendon 70. That is, the first anchor section 12 and the second anchor section 14 are squeezed together with the pliers 40 so that the tines 24 of the first anchor section 12 extend through the overlapped section 76 of tendon 70, i.e. through both the proximal free end 72 of the Achilles tendon 70 and the distal free end 74 of the Achilles tendon 70. The tines 24 of the first anchor section 12 are then embedded in the plastic material 32 of the second anchor section 14, which plastically deforms to receive the tines 24, to secure the proximal free end 72 of the Achilles tendon 70 to the distal free end 74 of the Achilles tendon 70. The anchor 10 is left in position after surgery to hold the Achilles tendon 70 together. In some embodiments, the anchor 10 is formed from a biocompatible material that allows the anchor 10 to be permanently retained in the surgical site. In other embodiments, the anchor 10 is formed from a material that absorbs into the body after surgery. For example, the anchor 10 may be biodegradable and/or bio-absorbable. In some embodiments, the anchor 10 is formed from a biocompatible material that allows tissue ingrowth. The anchor 10 can be formed in different sizes and shapes to accommodate patient anatomy and pathology.

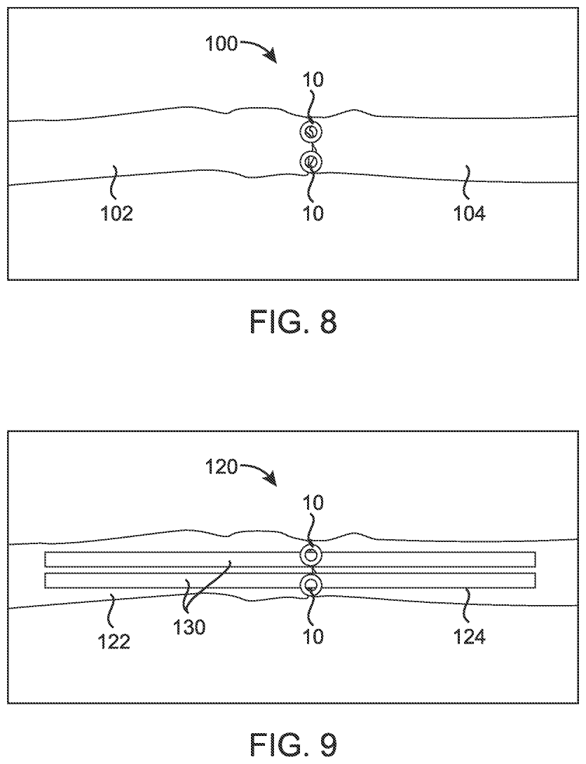

FIG. 8 illustrates another embodiment for repairing a torn tendon 100 using an anchor 10. The torn tendon 100 includes a first free end 102 and a second free end 104. The first free end 102 and the second free end 104 are positioned adjacent one another to form an adjoined section of tendon. The anchor 10 is secured to both the first free end 102 and the second free end 104. That is, a portion of the anchor 10 is secured to the first free end 102 and another portion of the anchor 10 is secured to the second free end 104 so that the first free end 102 and the second free end 104 are held together by the anchor 10. In the illustrated embodiment, two anchors 10 are used to secure the first free end 102 to the second free end 104. It will be appreciated that any number of anchors 10 may be used to secure the first free end 102 to the second free end 104. The number of anchors 10 utilized may depend on a size, for example, width, of the ligament.

FIG. 9 illustrates yet another embodiment for repairing a torn tendon 120 using an anchor 10. The torn tendon 120 includes a first free end 122 and a second free end 124. The first free end 122 and the second free end 124 are positioned adjacent one another to form an adjoined section of tendon. Suture tape 130 is extended over the adjoined section of tendon from the first free end 122 to the second free. In the illustrated embodiment, two pieces of suture tape 130 are used to secure the first free end 102 to the second free end 104. It will be appreciated that any number of pieces of suture tape 130 may be used to secure the first free end 102 to the second free end 104. The number of pieces of suture tape 130 utilized may depend on a size, for example, width, of the ligament. The suture tape 130 may be applied to only one side of the adjoined section of tendon. In other embodiments, the suture tape 130 is applied to both sides of the adjoined section of tendon. In some embodiments, the suture tape 130 is formed from a biocompatible material that allows the suture tape 130 to be permanently retained in the surgical site. In other embodiments, the suture tape 130 is formed from a material that absorbs into the body after surgery. For example, the suture tape 130 may be biodegradable and/or bio-absorbable. In some embodiments, the suture tape 130 is formed from a biocompatible material that allows tissue ingrowth. The suture tape 130 can be formed in different sizes and shapes to accommodate patient anatomy and pathology.

The anchor 10 is secured to both the first free end 122 and the second free end 124 over the suture tape 130. That is, a portion of the anchor 10 is secured to the first free end 122 and another portion of the anchor 10 is secured to the second free end 124 so that the first free end 122 and the second free end 124 are held together by the anchor 10. In the illustrated embodiment, two anchors 10 are used to secure the first free end 122 to the second free end 124. It will be appreciated that any number of anchors 10 may be used to secure the first free end 122 to the second free end 124. The number of anchors 10 utilized may depend on a size, for example, width, of the ligament.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

1. A method of repairing a tendon having a first free end and a second free end, the method comprising:
positioning the first free end of tendon adjacent the second free end of tendon to form an adjoined section of tendon,
positioning a first anchor section against a first side of the adjoined section of tendon,
positioning a second anchor section against a second side of the adjoined section of tendon so that the second anchor section is aligned with the first anchor section,
securing the first anchor section to the second anchor section to secure the first free end of tendon to the second free end of tendon.

2. The method of claim 1, wherein securing the first anchor section to the second anchor section further comprises securing a fastening mechanism of the first anchor section to the second anchor section.

3. The method of claim 2, wherein securing a fastening mechanism of the first anchor section to the second anchor section further comprises securing a tine of the first anchor section to the second anchor section.

4. The method of claim 3, wherein securing a tine of the first anchor section to the second anchor section further comprises embedding the tine of the first anchor section into the second anchor section.

5. The method of claim 1, wherein the second anchor section is configured to plastically deform when the first anchor section is secured to the second anchor section.

6. The method of claim 1, wherein securing the first anchor section to the second anchor section further comprising extending a portion of the first anchor section through the adjoined section of tendon.

7. The method of claim 6, wherein extending a portion of the first anchor section through the adjoined section of tendon further comprises extending the portion of the first anchor section through the first free end of tendon and the second free end of tendon.

8. The method of claim 6, further comprising extending the portion of the first anchor section through the second anchor section.

9. The method of claim 1, wherein securing the first anchor section to the second anchor section further comprises squeezing the first anchor section and the second anchor section together.

10. An anchor for repairing a tendon having a first free end positioned adjacent a second free end to form an adjoined section of tendon, the anchor comprising:
a first anchor section having a fastening mechanism, wherein the first anchor section is configured to position on a first side of the adjoined section of tendon,
a second anchor section configured to position on a second side of the adjoined section of tendon,
wherein the fastening mechanism of the first anchor section is configured to secure to the second anchor section to secure the first free end of tendon to the second free end of tendon.

11. The anchor of claim 10, wherein the fastening mechanism is a tine that is configured to secure to the second anchor section.

12. The anchor of claim 11, wherein the second anchor section is formed from a plastic material and the tine is configured to embed in the plastic material.

13. The anchor of claim 12, further comprising a plurality of tines that embed in the plastic material.

14. The anchor of claim 10, wherein the first anchor section and the second anchor section are formed as rings.

15. The anchor of claim 14, wherein the first anchor section includes:
a ring shaped body, and
a plurality of fastening mechanisms extending from the ring shaped body.

16. The anchor of claim 10, wherein the fastening mechanism of the first anchor portion is configured to extend through the adjoined section of tendon.

17. An anchor for repairing a tendon having a first free end positioned adjacent a second free end to form an adjoined section of tendon, the anchor comprising:
a first anchor section, wherein the first anchor section is configured to position on a first side of the adjoined section of tendon,
a second anchor section formed from a plastic material and configured to position on a second side of the adjoined section of tendon,
wherein a portion of the first anchor section is configured to embed in the second anchor section to secure the first free end of tendon to the second free end of tendon.

18. The anchor of claim 17, wherein a plurality of portions of the first anchor section are configured to embed in the second anchor section.

19. The anchor of claim 17, wherein the portion of the first anchor section is configured to extend through the adjoined section of tendon.

20. The anchor of claim 17, wherein the first anchor section and the second anchor section are formed as rings.
